(19) United States
(12) Patent Application Publication
(10) Pub. No.: US 2024/0226531 A1
(43) Pub. Date: Jul. 11, 2024

(54) Universal Caps for Male and Female Connectors with an Inner Shaft and Outer Threaded Housing

(71) Applicant: Becton, Dickinson and Company, Franklin Lakes, NJ (US)

(72) Inventors: Vivek K T, Bengaluru (IN); Sandip Satishsonar, Dist-Sangli (IN); Tharanath E, Bengaluru -96 (IN); Anandhakannan Rajugovindasamy, Coimbatore (IN)

(21) Appl. No.: 18/152,932

(22) Filed: Jan. 11, 2023

(51) Int. Cl. A61M 39/20; A61M 1/00; A61M 39/10

(52) U.S. Cl. CPC A61M 39/20; A61M 1/912; A61M 39/10; A61M 2039/1033; A61M 2039/1083; A61M 2039/1088

(57) ABSTRACT

A universal cap for a first connector or a second connector of different types includes an inner housing having a base with an open first end, a closed second end, and a sidewall extending therebetween. The inner housing also includes a shaft with a first end and a second end opposite the first end, which is connected to the closed second end of the base. The cap also includes an outer housing positioned around the shaft of the inner housing having an open first end, an open second end, and a sidewall extending therebetween. An inner surface of the sidewall of the outer housing includes threads configured to engage corresponding threads of the first connector or the second connector. The cap also includes at least one absorbent member connected to the shaft configured to contact portions of the first connector or the second connector.

### BACKGROUND OF THE INVENTION

### Field of the Invention

The present disclosure relates generally to caps for medical connectors and, in particular, to a medical cap configured to be attached to at least two different types of connectors, such as to a male luer connector or a female luer connector.

### Description of Related Art

Vascular access devices (VADs) are commonly used medical devices, which can include intravenous (IV) catheters, such as peripheral catheters or central venous catheters. If not properly maintained or if exposed to a non-sterile environment, the VADs can become contaminated, sealed with blood clots, and/or can spread infection. Further, bacteria and other microorganisms may enter into a patient's vascular system from access hubs, ports, or valves upon connection to the VAD to deliver a fluid or pharmaceutical to a patient. Therefore, each access hub (or port/valve or connection) configured for attachment to a VAD is associated with some risk of transmitting a catheter related bloodstream infection (CRBSI) to a patient.

Many medical facilities implement sterile practices and protocols to ensure that VADs and access hubs or ports are used properly and do not become sealed or infected. These protocols often include sterilizing the access hubs, ports, and VADs, as well as flushing the catheter with a flush solution prior to use. Specifically, VAD standards of practice usually recommend flush procedures be performed after catheter placement, before fluid infusion, and before and after drug administration, blood sampling, transfusions, and/or administration of parenteral nutrition. Standards of practice can also require that access hubs, ports, and valves be capped with disinfection caps when not in use to prevent microbial ingress into the hub, port, or valve and to sterilize areas of the hub, port, or valve that contact the VAD. Disinfection caps are disposable cap devices that contain an amount of cleaning or disinfecting solution for sterilizing portions of the port, hub, and valve.

Access hubs and ports can have a variety of different types of male or female connectors for securing the hub or port to the VAD. Currently, practitioners often carry several types of caps with them so that they can cap the different types of hubs and ports that may be used for a particular patient. For example, caps for male needleless connectors and female needleless connectors, as well as IV and hemodialysis lines, use different connector designs and may require different caps. In some examples, there can be “male disinfecting cap devices” for disinfecting ISO594-2 type of female threaded fluid luer connectors and “female disinfecting cap devices” for disinfecting ISO594-2 type of male threaded fluid luer connectors.

Some examples of universal caps that fit on both male and female connectors are known. For example, U.S. Pat. No. 10,871,246, entitled “Universal Connector or Cap for Male and Female Threaded Fittings,” and U.S. Patent Appl. Pub. No. 2008/0177250A1, entitled “Nestable sterility-protecting caps for separated connectors”, which are incorporated herein by reference in their entireties, disclose caps including threaded protrusions that can engage both a male connector and a female connector. However, there is a need for simpler cap designs that can be manufactured inexpensively and efficiently. The universal caps of the present disclosure are configured to attach to both male and female medical connectors in a secure manner sufficient for preventing microbial ingress.

### SUMMARY OF THE INVENTION

According to an aspect of the present disclosure, a universal cap configured to engage at least a first connector and a second connector of different types includes an inner housing having a base with an open first end, a closed second end, and a sidewall extending therebetween. The inner housing also includes a shaft with a first end, a second end opposite the first end, which is connected to the closed second end of the base, and a sidewall extending between the first end and the second end of the shaft through the open first end of the base. The cap also includes an outer housing positioned around the shaft of the inner housing having an open first end, an open second end, and a sidewall extending therebetween. An inner surface of the sidewall of the outer housing includes threads configured to engage corresponding threads of the first connector or the second connector. The shaft of the inner housing is configured to move through the outer housing toward the first connector or the second connector. The cap also includes at least one absorbent member connected to the first end of the shaft configured to contact portions of the first connector or the second connector, when the first connector or the second connector is inserted through the open first end of the outer housing.

In accordance with an embodiment of the present invention, a universal cap configured to engage at least a first connector and a second connector of different types, includes an inner housing comprising a base having an open first end, a closed second end, a sidewall extending therebetween. The cap includes a shaft having a first end and a second end opposite the first end, which is connected to the closed second end of the base, an outer housing positioned around the shaft of the inner housing comprising an open first end, an open second end, and a sidewall extending therebetween. An inner surface of the sidewall of the outer housing includes threads configured to engage corresponding threads of the first connector or the second connector, and wherein the shaft of the inner housing is configured to move through the outer housing toward the first connector or the second connector; and at least one absorbent member connected to the first end of the shaft configured to contact portions of the first connector or the second connector, when the first connector or the second connector is inserted through the open first end of the outer housing.

In accordance with an embodiment of the present invention, the first connector is a male needleless connector, and wherein the shaft of the inner housing is configured to be secured to the male needleless connector by an interference or friction engagement between the shaft and an outer surface of the male needleless connector.

In accordance with an embodiment of the present invention, the male needleless connector is a male luer connector comprising a stem having a tapered outer surface, and wherein the shaft includes a tapered cavity sized to receive the stem of the male luer connector for securing the male luer connector to the cap.

In accordance with an embodiment of the present invention, the male luer connector further includes an annular shield extending about the stem, and wherein an open end of the annular shield contacts the open first end of the outer housing as the stem of the male luer connector is inserted through the open first end of the outer housing.

In accordance with an embodiment of the present invention, the outer housing has an inner diameter of less than about 14.0 mm so that the annular shield of the male luer connector does not insert into the outer housing.

In accordance with an embodiment of the present invention, the second connector is a female needleless connector, and wherein the threads on the inner surface of the outer housing are configured to engage corresponding threads of the female needleless connector for securing the female needleless connector to the cap.

In accordance with an embodiment of the present invention, the female needleless connector is a female luer connector comprising a body defining a tapered cavity having a distal end configured to contact the first end of the shaft of the inner housing.

In accordance with an embodiment of the present invention, the outer housing is sized to receive the female luer connector having a maximum outer diameter of about 9.0 mm to about 11.0 mm.

In accordance with an embodiment of the present invention, the inner housing and/or the outer housing includes a rigid thermoplastic polymer, such as polyester, polycarbonate, polypropylene, polyethylene, polyethylene terephthalate, or acrylonitrile butadiene styrene.

In accordance with an embodiment of the present invention, the shaft includes a tapered cavity extending inwardly from the first end of the shaft sized to receive a stem of a male luer connector.

In accordance with an embodiment of the present invention, the inner housing includes a substantially opaque polymer material.

In accordance with an embodiment of the present invention, the shaft includes at least one protrusion configured to rotatably engage threads of the outer housing, such that rotation of the inner housing relative to the outer housing draws the shaft of the inner housing through the outer housing.

In accordance with an embodiment of the present invention, the at least one protrusion includes a plurality of lugs configured to rotatably engage the threads on the inner housing of the sidewall of the outer housing.

In accordance with an embodiment of the present invention, the cap further includes an annular seal about the shaft of the inner housing configured to seal against an inner surface of the sidewall of the outer housing preventing cleaning solution from passing through the open second end of the outer housing.

In accordance with an embodiment of the present invention, the seal is integral with an outer surface of the shaft.

In accordance with an embodiment of the present invention, the seal includes an O-ring positioned about the shaft.

In accordance with an embodiment of the present invention, the inner housing is a single-molded part.

In accordance with an embodiment of the present invention, the outer housing is configured to move from an initial position, in which the open second end of the outer housing is closer to the open first end of the base than to the closed second end of the base, and a final position, in which the open second end of the outer housing is closer to the closed second end of the base than to the open first end of the base.

In accordance with an embodiment of the present invention, the outer housing includes a transparent, substantially transparent, or translucent material.

In accordance with an embodiment of the present invention, the inner housing can be seen through the transparent, substantially transparent, or translucent material of the outer housing.

In accordance with an embodiment of the present invention, the at least one absorbent member is positioned to cover an opening of the first connector or the second connector when the first connector or the second connector is inserted through the open first end of the outer housing.

In accordance with an embodiment of the present invention, the at least one absorbent member includes a cylindrical absorbent member and a tubular absorbent member positioned around the cylindrical absorbent member.

In accordance with an embodiment of the present invention, the shaft includes a cavity extending inward from the first end of the shaft, wherein the cylindrical absorbent member is at least partially received within the cavity, and wherein the tubular absorbent member encloses the first end of the cavity and the cylindrical absorbent member, such that a portion of an inner surface of the tubular absorbent member contacts an outer surface of the shaft.

In accordance with an embodiment of the present invention, the tubular absorbent member includes at least one opening sized to receive a lug extending outward from the sidewall of the shaft.

In accordance with an embodiment of the present invention, the cylindrical absorbent member and/or the tubular absorbent member includes a porous foam having a thermoplastic elastomeric material.

In accordance with an embodiment of the present invention, the tubular absorbent member is shaped to clean and/or disinfect a distal portion and outer surface of a female connector and the cylindrical absorbent member is shaped to clean and/or disinfect a tip and outer surface of a stem of a male connector.

In accordance with an embodiment of the present invention, insertion of the first connector through the open first end of the outer housing causes the first connector to contact the cylindrical absorbent member, and wherein contact between the first connector and the cylindrical absorbent member mechanically removes objects from outer surface(s) of the first connector.

In accordance with an embodiment of the present invention, insertion of the second connector through the open first end of the outer housing causes the second connector to contact an outer surface of the tubular absorbent member, and wherein contact between the second connector and the tubular absorbent member mechanically removes objects from outer surface(s) of the second connector.

In accordance with an embodiment of the present invention, a cleaning or disinfecting solution is absorbed by the cylindrical absorbent member and the tubular absorbent member.

In accordance with an embodiment of the present invention, the cleaning or disinfecting solution includes an antimicrobial, anti-fungal, antibacterial, or antiviral composition.

In accordance with an embodiment of the present invention, the cleaning or disinfecting solution includes about 0.5% to about 3.5% chlorhexidine gluconate, about 60% to 85% isopropyl alcohol (IPA), or combinations thereof.

In accordance with an embodiment of the present invention, the cap further including a removable protective cover attached to the open first end of the outer housing for protecting an interior of the outer housing and the at least one absorbent member prior to use.

In accordance with an embodiment of the present invention, the protective cover includes a film with adhesive on a first side of the film for removably mounting the first side of the film to the open second end of the housing.

In accordance with an embodiment of the present invention, the protective cover is substantially impervious to air to prevent the absorbent member from drying prior to use.

In accordance with an embodiment of the present invention, the protective cover is connected to the open second end of the housing by heat sealing.

### BRIEF DESCRIPTION OF THE DRAWINGS

FIG. 1A is a cross-sectional view of an exemplary male connector, as is known in the prior art.

FIG. 1B is an example of a female connector including a septum with a slit, as is known in the prior art.

FIG. 2 is a perspective view of a cross-section of a universal cap, according to an aspect of the present disclosure.

FIG. 3A is a perspective view of an inner housing and absorbent members of the universal cap of FIG. 2.

FIG. 3B is an enlarged perspective view of a portion of the inner housing and absorbent members of FIG. 3A.

FIGS. 4A and 4B are enlarged perspective views of portions of the inner housing of the universal cap of FIG. 2.

FIG. 5A is a front view showing the universal cap of FIG. 2 and a male connector in an initial position, according to an aspect of the present disclosure.

FIG. 5B is a cross-sectional view of the universal cap and the male connector of FIG. 5A, in the initial position.

FIG. 5C is a front view showing the universal cap and the male connector of FIG. 5A in an engaged position, according to an aspect of the present disclosure.

FIG. 5D is a cross-sectional view of the universal cap and the male connector of FIG. 5A, in the engaged position.

FIG. 6A is a front view showing the universal cap of FIG. 2 and a female connector in an initial position, according to an aspect of the present disclosure.

FIG. 6B is a cross-sectional view of the universal cap and the female connector of FIG. 6A, in the initial position.

FIG. 6C is a front view showing the universal cap and the female connector of FIG. 6A in an engaged position, according to an aspect of the present disclosure.

FIG. 6D is a cross-sectional view of the universal cap and the male connector of FIG. 6A, in the engaged position.

FIG. 7A is a perspective view of a cross-section of another example of a universal cap, according to an aspect of the present disclosure.

FIG. 7B is a perspective view of a cross-section of the universal cap of FIG. 7A and a female connector, according to an aspect of the present disclosure.

FIG. 7C is a perspective view of a cross-section of the universal cap of FIG. 7A and a male connector, according to an aspect of the disclosure.

FIG. 8 is a perspective view of the universal cap of FIG. 7A including a protective cover, according to an aspect of the present disclosure.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

The present disclosure is directed to caps 10, 210 configured to be connected to a medical connector 110, 112, such as an access hub, port, or valve for a VAD, to prevent the connector, port, or VAD from being contaminated by, for example, microbes, debris, or other contaminants. In some examples, the caps 10, 210 can be configured to clean or disinfect the connector 110, 112 or port, ensuring that the connector 110, 112 or port remains sterile prior to use. The caps 10, 210 can be configured to remain in place on a connector 110, 112 or port for at least seven days, which is a maximum time of recommended use permitted by many medical facility sterile practice guidelines. The caps 10, 210 can be configured to engage or be connected to different sizes, configurations, or types of medical connectors 110, 112. For example, the caps 10, 210 can be configured to engage or be connected to both a male connector 110 and a female connector 112.

### Male and Female Luer Connectors

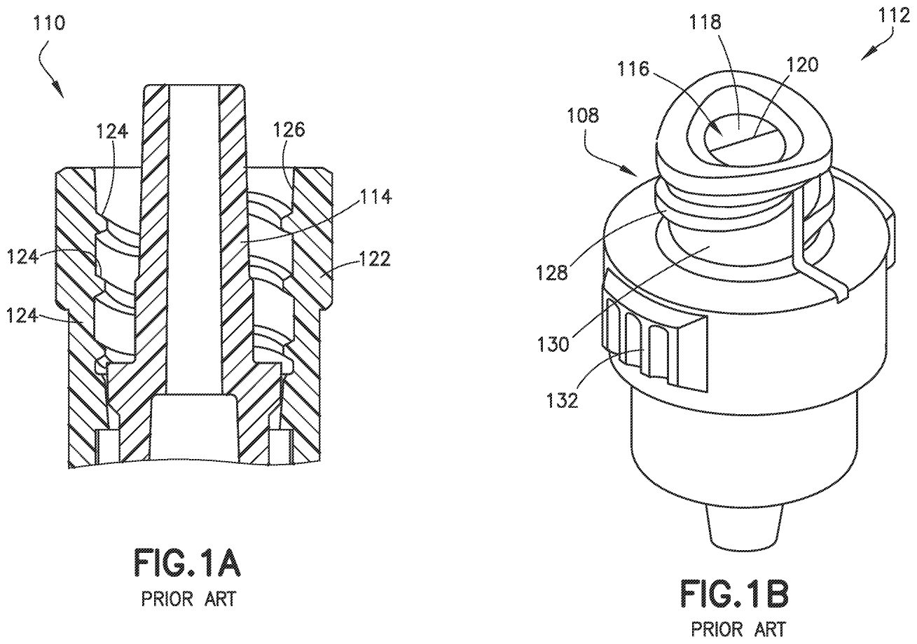

As used herein, a “male connector” refers to a connector 110 comprising an elongated member, such as a tubular member or stem 114, configured to be inserted in a tube or opening having an inner diameter that is larger than an outermost diameter of the male connector 110. An exemplary male connector 110 is shown in FIG. 1A. A “female connector” refers to a connector 112 comprising an opening or port 116 that is configured to receive an elongated member or tubular member of another object or device in order to connect the object or device to the female connector 112. The female connector 112 can have a cover or septum 118 over the opening 116. An exemplary female connector 112 including a septum 118 with a slit 120 is shown in FIG. 1B.

In some examples, the cap 10 of the present disclosure is configured to engage both a male luer connector 110 and a female luer connector 112. For example, the cap 10 can be configured to receive a female luer connector 112 having an outer diameter of at least 9.0 mm, at least 10.0 mm, or at least 12.5 mm. The cap 10 can also be sized to receive a male luer connector 110 having an outer diameter of from about 8.0 mm to about 14.0 mm.

As used herein, a “luer connector” refers to a connector that includes a tapered portion (i.e., a luer taper) for creating a friction engagement between a tapered stem 114 or elongated member of a male luer connector 110 and a tapered cavity. Specifically, the male luer connector 110 can include a tapered stem 114 or elongated member having a tapered outer surface, such as an outer surface having a taper of about 6%. The female luer connector 112 can include a tapered cavity configured to receive and engage the tapered stem 114 or elongated member to connect the male luer connector 110 to the female luer connector 112.

In order to secure the male and female connectors 110, 112 together, in some examples, the connectors 110, 112 can include engaging structures, such as threads, for drawing the connectors 110, 112 together. For example, as shown in FIG. 1A, the male luer connector 110 can include an annular shield 122 extending about the tapered stem 114 or elongated member. The annular shield 122 can include threads 124 on an inner surface 126 of the shield 122 configured to engage corresponding threads 128 on an outer surface 130 of the female luer connector 112. More specifically, as shown in FIG. 1B, the female luer connector 112 can include threads 128 extending from the outer surface 130 positioned to engage the threads 124 on the inner surface 126 of the annular shield 122 of the male luer connector 110. Twisting the female connector 112 relative to the male connector 110 causes the corresponding threads 124, 128 to engage, which draws the connectors 110, 112 together, such that the tapered stem 114 or elongated member of the male luer connector 110 moves through the opening 116 of the female connector 112. In some examples, the female connector 112 can also include vertical ridges 132 near a proximal end of the female connector 112, which can be used to manipulate the female connector 112 making it easier to twist the female connector 112 compared to another connector or device. As previously described, the caps 10 of the present disclosure are configured to cover both the male luer connector 110 (shown in FIG. 1A) and the female luer connector 112 (shown in FIG. 1B).

### Universal Caps for Male and Female Connectors

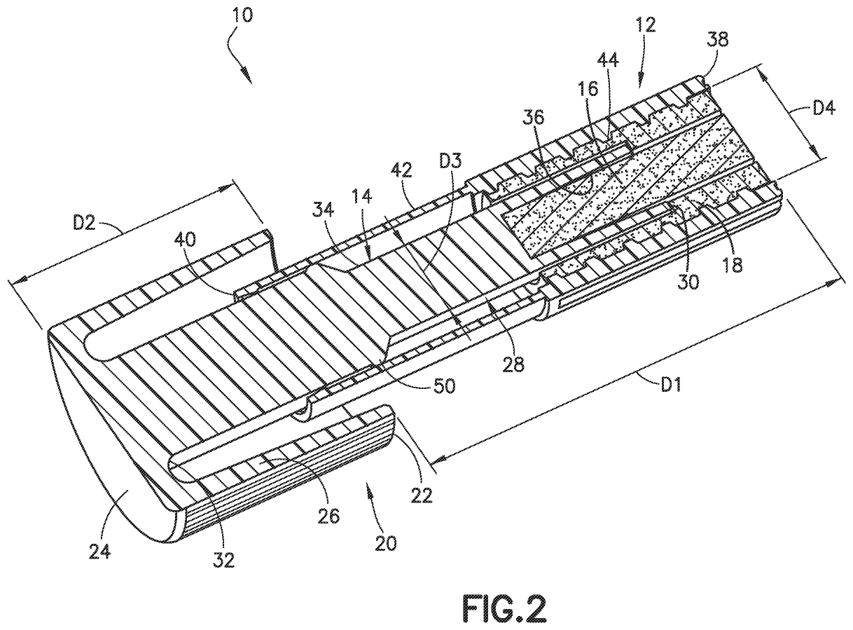

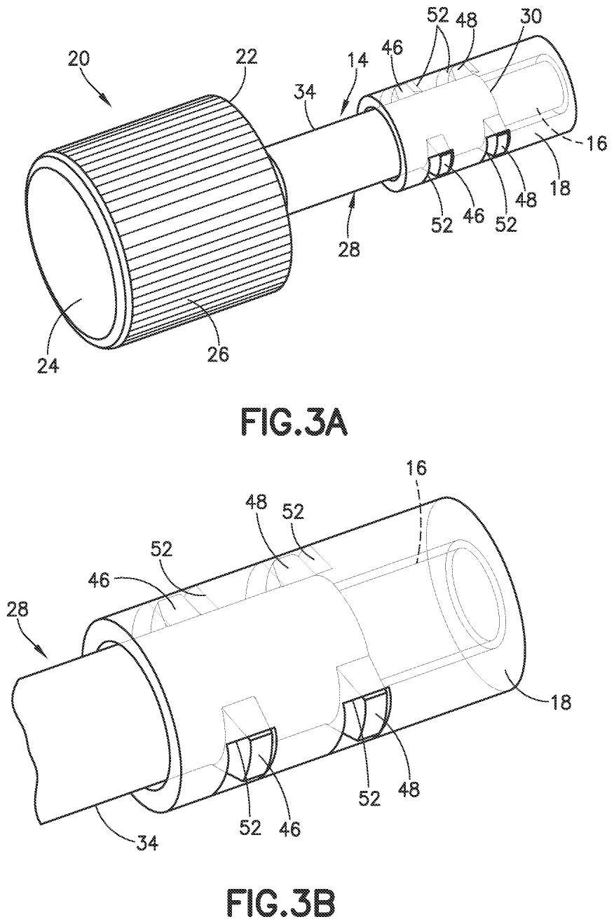

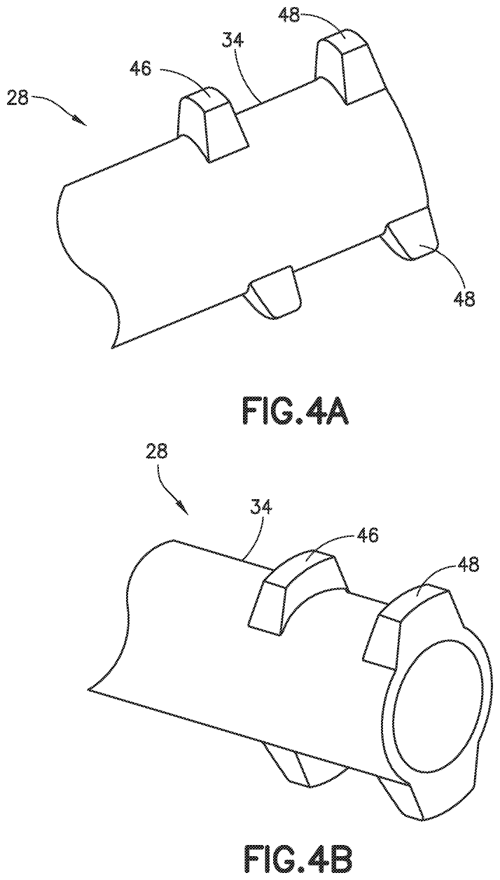

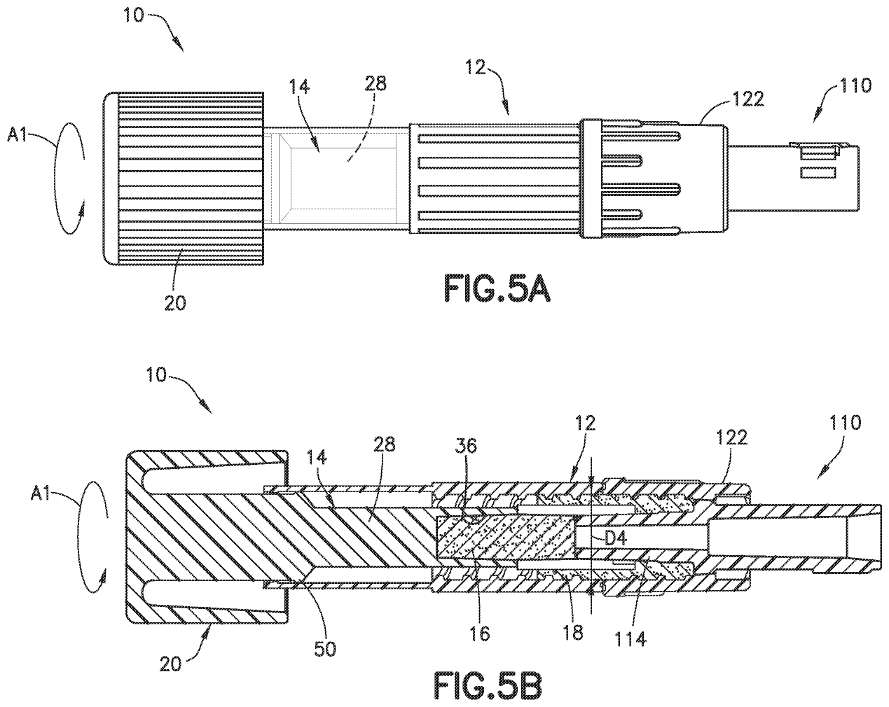

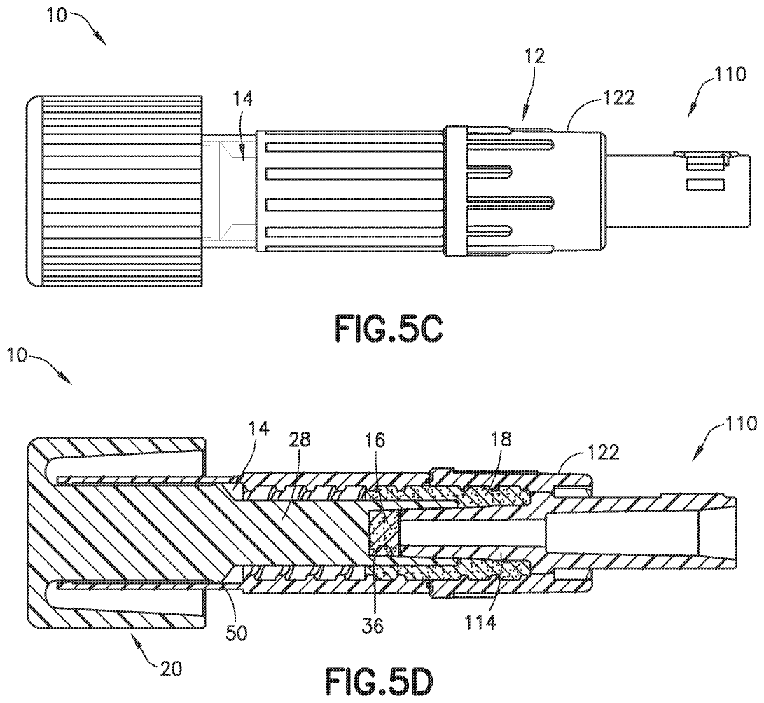

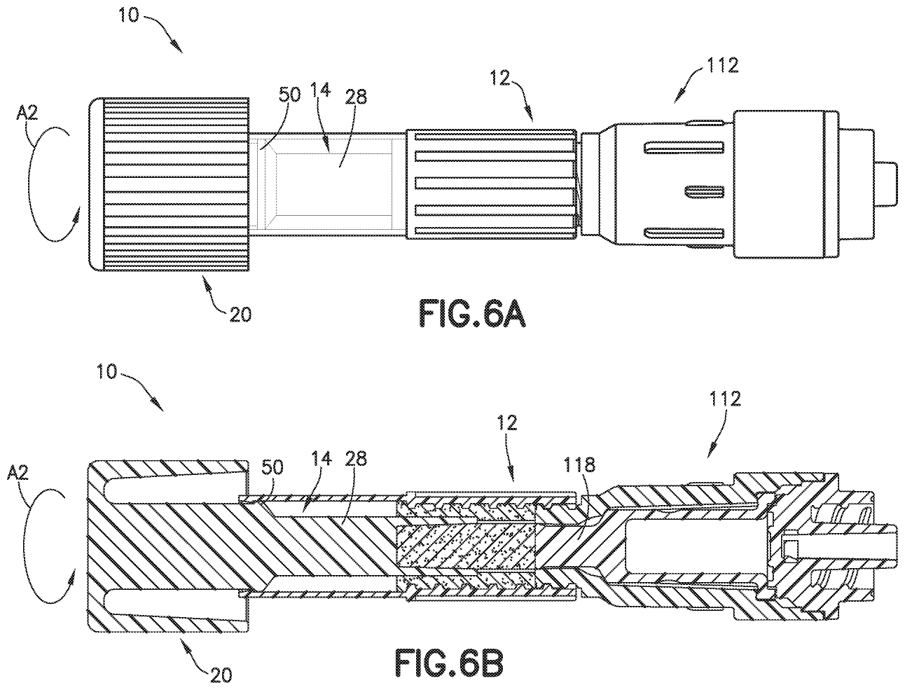

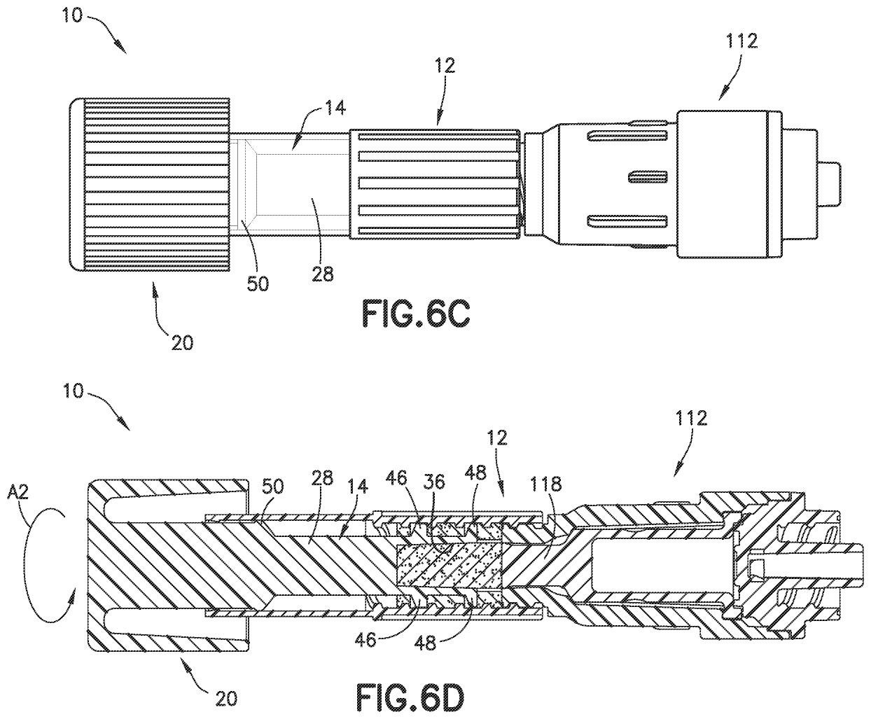

FIGS. 2-6D show an exemplary universal cap 10 configured to be connected to different types of connectors, such as to both the male luer connector 110 and the female luer connector 112. Specifically, FIG. 2 is a perspective view of a cross-section of the cap 10. FIGS. 3A and 3B are enlarged perspective views showing an absorbent support or absorbent member of the cap 10 configured to contact portions of the connectors 110, 112 for scrubbing (e.g., mechanically removing microbes, dust, debris, and other particles) from surfaces of the connectors 110, 112. FIGS. 4A and 4B are perspective views showing protrusions or lugs of an inner portion or housing of the cap 10, which are configured to engage corresponding threads on an outer portion or housing of the cap. FIGS. 5A-5D are front and cross-sectional views showing the universal cap 10 of the present disclosure connected to a male connector 110, such as a male needleless connector or a male luer connector. FIGS. 6A-6D are front and cross-sectional views showing the universal cap 10 of the present disclosure connected to a female connector 112, such as to a female needleless connector or a female luer connector.

There are numerous different commercially available medical devices, such as hubs, ports, and valves, which can include different variations of the male or female connectors 110, 112. The cap 10 of the present disclosure can be configured to adapt or deform so that it can be secured to numerous different types and sizes of these connectors. For example, the cap 10 of the present disclosure can be configured to attach to male or female Luer-Lok™ connectors by Becton, Dickinson and Company. The cap 10 of the present disclosure can also be configured to cover different connector designs including, without limitation, the BD Q-Syte™, BD MaxZero™, BD MaxPlus™, and SmartSite™ needle free connectors by Becton, Dickinson and Company. The caps 10 can also be configured to be connected to male and/or female connectors by other manufactures including, without limitation, MicroClave® connectors (ICU Medical Inc.) and Ultrasite® connectors (B. Braun Medical Inc.). In other examples, the cap 10 can be configured to be connect to one or more of the following commercially available male connectors: Kendall 2001NP; BD MP5303-C; ICU Med 12664-28; RyMed RYM-5307HPU; B. Braun 470108; Baxter 2C8537; Kawasumi IV-0094; Zyno B2-70071-D; B. Braun 470124; Baxter 2C7462; and Smith's Medical 536035.

As shown in FIG. 2, the universal cap 10 comprises an outer housing 12 and an inner housing 14 configured to move axially through the outer housing 12 as the male connector 110 or the female connector 112 is connected to the universal cap 10. In some examples, the inner housing 14 and/or the outer housing 12 are molded parts, which can be formed from various rigid moldable materials. For example, the outer housing 12 or the inner housing 14 can comprise a rigid thermoplastic polymer, such as polyester, polycarbonate, polypropylene, polyethylene, polyethylene terephthalate, or acrylonitrile butadiene styrene. In some examples, the housings 12, 14 of the cap 10 can be molded parts made by injection molding processes, as are known in the art, formed from any of the previously described thermoplastic materials. In some examples, the thermoplastic material can be a durable material, such as a material having a shore hardness D value of less than or equal to 60 shore D. Alternatively, the cap 10 can be formed from a more flexible thermoplastic material, such as a material having a shore hardness A value less than or equal to 80 shore A.

In some examples, as shown in the figures, the inner housing 14 is formed from or includes an opaque or substantially opaque material. As used herein, an opaque or substantially opaque material can refer to a material having a light transmittance of less than about 20%, less than about 10%, or, for example, between about 5% and about 10%. By contrast, the outer housing 12 can include or can be formed from a material that is transparent, substantially transparent, or translucent, so that the practitioner can see portions of the inner housing 14 that are covered by the outer housing 12 during use of the cap 10. For example, the outer housing 12 can be formed from a material having a light transmittance of at least 50%, at least 75%, at least 90% or, for example, between about 90% and about 95%.

The universal cap 10 also includes one or more absorbent supports, members, or devices, referred to herein as absorbent members 16, 18, connected a top portion of the inner housing 14. For example, as described in further detail herein, the absorbent members 16, 18 can be positioned and/or configured to contact portions of the male connector 110 or the female connector 112, when the male connector 110 or the female connector 112 is inserted into the outer housing 12 for cleaning and disinfecting surfaces of the connectors 110, 112.

In some examples, the inner housing 14 of the cap 10 comprises a base 20 having an open first or top end 22, a closed second or bottom end 24, and a sidewall 26 extending therebetween. The sidewall 26 can have a length D2 of about 15 mm to about 30 mm. In some examples, as shown in FIG. 2, an outer surface of the base 20 is textured to facilitate gripping and rotating the base 20 during use. For example, as shown in FIG. 2, the outer surface of the base 20 can include axial ridges or protrusions to assist the practitioner in grasping, manipulating, twisting, or rotating the base 20. The inner housing 14 also includes a shaft 28 having a first or top end 30, a second or bottom end 32 opposite the top end 30, which is connected to the closed bottom end 24 of the base 20, and a sidewall 34 extending between the top end 30 and the bottom end 32 of the shaft 28. As shown in FIG. 2, the shaft 28 extends through the open top end 22 of the base 20. In some particular examples, the shaft 28 can protrude beyond the top end 22 of the base 20 by a distance D1 of from about 10 mm to about 30 mm or, preferably, from about 15 mm to about 20 mm. The shaft 28 has a diameter D3 of about 5 mm to about 15 mm or, preferably, of about 8 mm to 12 mm.

As shown most clearly in FIGS. 5B and 5D, the shaft 28 includes a cavity 36, such as a tapered cavity, extending inwardly from the top end 30 of the shaft 28. The cavity 36 is sized to receive the stem 114 of the male connector 110 for providing a friction engagement between an outer surface of the male connector 110 and an inner surface of the shaft 28.

The outer housing 12, which is positioned at least partially around the shaft 28 of the inner housing 14, comprises an open first or top end 38, an open second or bottom end 40, and a sidewall 42 extending between the top end 38 and the bottom end 40 of the outer housing 12. The outer housing 12 is configured to move axially along the shaft 28 into the base 20 as the male connector 110 or the female connector 112 is attached to the cap 10. For example, the outer housing 12 can be configured to move from an initial position (shown in FIGS. 5A, 5B, 6A, and 6B), in which the open bottom end 40 of the outer housing 12 is closer to the open top end 22 of the base 20 than to the closed bottom end 24 of the base 20, and a final or engaged position (shown in FIGS. 5C, 5D, 6C, and 6D), in which the open bottom end 40 of the outer housing 12 is closer to the closed bottom end 24 of the base 20 than to the open top end 22 of the base 20.

In some examples, the outer housing 12 includes engaging structures for securing the male connector 110 or the female connector 112 to the outer housing 12 and also for supporting the shaft 28 as it moves through the outer housing 12. For example, an inner surface of the sidewall 42 of the outer housing 12 can include threads 44 (e.g., a single helical thread extending over the inner surface of the sidewall 42 between the top end 38 and the bottom end 40 of the outer housing 12). The threads 44 can be configured to engage corresponding threads 128 of the female connector 112. The threads 44 can also be configured to rotatably engage protrusions (e.g., threads, lugs, ridges, and similar structures) extending from the shaft 28 so that the shaft 28 of the inner housing 14 moves through the outer housing 12 towards the male connector 110 or the female connector 112. For example, the shaft 28 of the inner housing 14 can include one or more lugs 46, 48 configured to rotatably engage the threads 44 on the inner surface of the sidewall 42 of the outer housing 12. In particular, as shown most clearly in FIGS. 3A-4B, the shaft 28 can include four total lugs (e.g., two upper lugs 48 and two lower lugs 46). As shown in FIGS. 3A and 3B, the upper lugs 48 can be axially aligned with the lower lugs 46. The lugs 46, 48 can be generally trapezoidal in shape having a wide edge connected to the outer surface of the sidewall 34 of the shaft 28, a narrow edge opposite the wide edge, and sloped, angled, or oblique sides extending between the wide edge and the narrow edge. The lugs 46, 48 can be the same size and shape or different sizes and/or shapes. For example, as shown in FIG. 4B, the lower lugs 46 can have arcuate length that is longer than the upper lugs 48.

In some examples, the shaft 28 also includes an annular seal 50 about the shaft 28 of the inner housing 14 configured to seal against an inner surface of the sidewall 42 of the outer housing 12 preventing the cleaning or disinfecting solution contained by the absorbent members 16, 18 from passing through the open bottom end 40 of the outer housing 12 as the male connector 110 or the female connector 112 is being connected to the cap 10. As shown in FIG. 2, in some examples, the seal 50 is integral with an outer surface of the shaft 28. For example, the seal 50 can be an annular lip, ridge, or similar structure that is molded onto the outer surface of the shaft 28. In other examples, as described in further detail herein, the seal 50 can be a separate structure, such as an O-ring, attached to the shaft 28 for providing the seal 50 between the outer surface of the shaft 28 and the inner surface of the outer housing 12.

As previously described, the universal caps 10 disclosed herein are configured to connect to and clean or disinfect surfaces of both the male connector 110 and the female connector 112. For example, the cavity 36 of the shaft 28 can be sized to receive the stem 114 of the male connector 110 for securing the male connector 110 to the cap 10. Also, the annular shield 122 of the male connector 110 can contact the open top end 38 of the outer housing 12 as the stem 114 is inserted through the open top end 38 of the outer housing 12. Accordingly, the outer housing 12 can have an inner diameter D4 (shown in FIG. 2) of less than about 15.0 mm so that the annular shield 122 of the male connector 110 does not insert into the outer housing 12.

The universal cap 10 disclosed herein also engages the female connector 112, such as a female needleless connector or female luer connector. For example, the threads 44 on the inner surface of the outer housing 12 can be configured to engage corresponding threads 128 of the female connector 112 for securing the female connector 112 to the universal cap 10. Accordingly, the outer housing 12 can be sized to receive conventional female luer connectors having accepted dimensions, as known in the art. For example, the outer housing 12 of the cap 10 can be sized to receive a female connector 112 having a maximum outer diameter of about 9.0 mm to about 11.0 mm.

The universal cap 10 also includes the one or more absorbent members, such as the cylindrical absorbent member 16 and the tubular absorbent member 18, configured to contact outer surfaces of the male connector 110 or the female connector 112. The absorbent members 16, 18 can be positioned to cover an opening of the male connector 110 or the female connector 112 when the male connector 110 or the female connector 112 is inserted through the open top end 38 of the outer housing 12. The absorbent members 16, 18 can also be configured to contact or press against outer surfaces of the connectors 110, 112 for removing particles from such surfaces.

In some examples, the absorbent member 16, 18 comprise a thermoplastic elastomer, such as polypropylene, polyethylene, or synthetic or natural rubber (e.g., isoprene). For example, the absorbent members 16, 18 can be a porous foam or sponge capable of absorbing the cleaning or disinfecting solution and releasing the cleaning or disinfecting solution when compressed. An exemplary foam material for the absorbent members 16, 18 can be a Plastazote® foam, which is an engineered polymer foam by Zotefoams PCL. Desirably, the absorbent member(s) 16, 18 can be formed from abrasive materials configured to contact surfaces of the male connector 110 and the female connector 112 for scrubbing the surfaces to mechanically remove particles, such as microbes, dirt, dust, and other debris, from surfaces of the connectors 110, 112. Further, a porosity of the absorbent members 16, 18 should be optimized so that the material is abrasive enough to scrub or mechanically remove objects from surfaces of the connectors 110, 112, while, at the same time, limiting ingress of cleaning or disinfecting solution into portions of the connectors 110, 112.

In some examples, as previously described, the universal cap 10 comprises multiple absorbent members, such as an absorbent member configured to contact surfaces of the male connector 110 and another absorbent member configured to contact surfaces of the female connector 112. For example, as shown in FIGS. 3A and 3B, the cap 10 can include the cylindrical absorbent member 16, which is partially received within the cavity 36 of the shaft 28. The cylindrical absorbent member 16 can be positioned to contact the distal surface of the stem 114 for cleaning and/or disinfecting the distal end of the stem 114. Further, the cylindrical absorbent member 16 can be positioned to compress as the stem 114 is inserted into the outer housing 12 releasing the cleaning or disinfecting solution from the cylindrical absorbent member 16.

The cap 10 also includes the tubular absorbent member 18, which is positioned around a top portion of the shaft 28 and around the cylindrical absorbent member 16. The tubular absorbent member 18 can be positioned to contact, clean, and disinfect the annular outer surface of the stem 114 of the male connector 110 as the male connector 110 is inserted through the outer housing 12. The tubular absorbent member 18 can also be configured to contact a distal surface of the female connector 112 as the female connector is inserted in the outer housing 12. Specifically, the distal end of the female connector 112 can contact and press against the tubular absorbent member 18, causing the tubular absorbent member 18 to compress and release the cleaning or disinfecting solution into the outer housing 12. As shown in FIGS. 3A and 3B, the tubular absorbent member 18 can also include one or more openings 52 sized to receive the lugs 46, 48 of the shaft 28 so that the lugs 46, 48 can contact the threads 44 of the outer housing 12.

In some examples, the absorbent members 16, 18 can be provided (e.g., presoaked) with the cleaning or disinfecting solution. For example, the cleaning or disinfecting solution can be an antimicrobial, anti-fungal, antibacterial, or antiviral solution that cleans and sterilizes surfaces of the connectors 110, 112. In some examples, the cleaning solution can be isopropyl alcohol (IPA), such as about 70% isopropyl alcohol (IPA). In other examples, the cleaning solution can be about 0.5% to about 3.5% chlorhexidine gluconate alone or in combination with about 70% IPA. A chlorohexidine composition may be beneficial because it has a slower evaporation rate than IPA and, therefore, provides a more persistent disinfectant activity after the cap 10 is removed from the connector 110, 112 and before the VAD is connected to the hub, port, or valve.

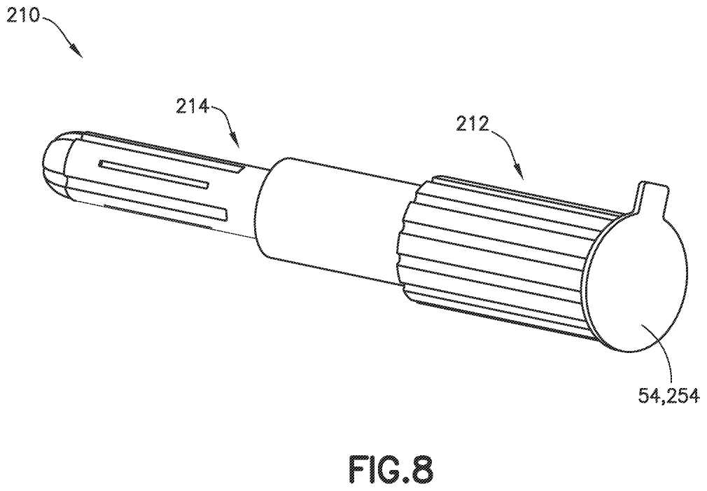

As shown in FIG. 8, the universal cap 10 can also include a removable protective cover 54 attached to the open top 38 of the outer housing 12 for protecting an interior of the outer housing 12 and the absorbent members 16, 18 contained therein prior to use. The protective cover 54 can comprise a sheet, such as a polymer film, with adhesive on a first side of the sheet for removably mounting the protective cover 54 to the open top of the outer housing 12. Alternatively, the protective cover 54 can be removably mounted to the open top 38 of the outer housing 12 of the cap 10 by heat sealing. The protective cover 54 can be slightly larger than the open top end 38 of the outer housing 12, so that the protective cover 54 can be easily grasped by the practitioner to remove the protective cover 54 from the cap 10 prior to use. The protective cover 54 can be formed from a material that is impervious or substantially impervious to air, so that the cleaning solution on the absorbent members 16, 18 does not evaporate or dry-out prior to use of the cap 10. Accordingly, the protective cover 54 increases a shelf life of the cap 10, as well as prevents microbes and other debris from collecting in the cap 10 prior to use.

### Methods of Connecting Universal Caps to Male and Female Connectors

As previously described, the caps 10 of the present disclosure are configured to be connected to a variety of different types of male connectors 110 and female connectors 112. FIGS. 5A-5D show the universal cap 10 being connected to a male connector 110. Specifically, in FIGS. 5A and 5B, the cap 10 and the male connector 110 are in an initial position with the shaft 28 of the inner housing 14 recessed within the outer housing 12 as far as possible away from the open top end 38 of the outer housing 12. Also, the annular shield 122 of the male connector 110 is in contact with the top end 38 of the outer housing 12. As shown in FIG. 5B, in the initial position, the stem 114 of the male connector 110 is in contact with the cylindrical absorbent member 16, but is spaced apart from the cavity 36 of the shaft 28.

In order to connect the cap 10 to the male connector 110, the practitioner rotates the inner housing 14 relative to the outer housing 12 (as shown by arrow A1). For example, the practitioner can grasp the outer housing 12 with one hand and the base 20 of the inner housing 14 in the other hand. The practitioner then twists or rotates the base 20 relative to the inner housing 14, which causes the shaft 28 to rotate within the outer housing 12. Rotation of the shaft 28 causes the lugs 46, 48 of the inner housing 14 to advance along the threads 44 of the outer housing 12, drawing the top end 30 of the shaft 28 towards the distal end of the stem 114. Movement of the shaft 28 towards the stem 114 causes the cylindrical absorbent member 16 to compress, releasing the cleaning or disinfecting solution, which contacts surfaces of the stem 114. Also, the annular outer surface of the stem 114 can contact an inner surface of the tubular absorbent member 18, which can mechanically remove any particles, such as microbes, dirt, dusk, and debris from the outer surfaces of the stem 114. As previously described, the seal 50 between the shaft 28 and the inner surface of the outer housing 12 retains the cleaning or disinfecting solution within the outer housing 12 preventing the solution from moving along the shaft 28 to the base 20 of the inner housing 14.

FIGS. 5C and 5D show the cap 10 and the male connector 110 in a fully engaged position. In this fully engaged position, the bottom end 40 of the outer housing 12 is inserted farther into the base 20 of the inner housing 14. Also, as shown in FIG. 5D, the stem 114 of the male connector 110 is inserted into the cavity 36 of the shaft 28, thereby forming a friction engagement between the stem 114 and the inner surface of the shaft 28. Further, the cylindrical absorbent member 16 is compressed into the cavity 36, such that the cleaning solution previously contained within the cylindrical absorbent member 16 is released into the outer housing 12 (i.e., into a space between the inner surface of the outer housing 12 and the surfaces of the shaft 28 and stem 114).

FIGS. 6A-6D show the universal cap 10 being connected to a female connector 112. As shown in FIGS. 6A and 6B, the cap 10 and female connector 112 are in the initial position with the shaft 28 of the inner housing 14 recessed within the outer housing 12, such that the top end 30 of the shaft 28 is spaced apart from the open top 38 of the outer housing 12 by a significant amount. Also, the female connector 112 is inserted a small distance through the open top end 38 of the outer housing 12, with corresponding threads 128 of the female connector 112 engaged with the threads 44 on the inner surface of the outer housing 12. Further, as shown in FIG. 6B, the female connector 112 can be in contact with the cylindrical absorbent member 16 and/or with the tubular absorbent member 18.

FIGS. 6C and 6D show the cap 10 fully engaged to the female connector 112. In order to advance the female connector 112 from the initial position to the fully engaged position, the practitioner rotates the inner housing 14 (as shown by arrow A2 in FIGS. 6A and 6B) relative to the outer housing 12 causing the shaft 28 to advance through the outer housing 12. In particular, the top end 30 of the shaft 28 moves through the outer housing 12 toward the distal end, surface, or septum 118 of the female connector 112. As the shaft 28 moves toward the female connector 112, the cylindrical absorbent member 16 compresses, releasing the cleaning or disinfecting solution, which contacts the outer surfaces and corresponding threads 128 of the female connector 112. In the fully engaged position, as shown in FIG. 6D, the distal end and septum 118 of the female connector 112 are proximate to the top end 30 of the shaft 28. However, unlike for the male connector 110, no portion of the female connector 112 is inserted into the cavity 36 of the shaft 28. Also, as in previous examples, the seal 50 on the shaft 28 contacts the inner surface of the outer housing 12, which retains the cleaning or disinfecting solution within the outer housing 12 (e.g., between the outer surface of the shaft 28, outer surfaces of the female connector 112, and the inner surface of the outer housing 12).

For either the male connector 110 or the female connector 112, the cap 10 can be removed from the connector 110, 112 by rotating the inner housing 12 and shaft in an opposite direction (i.e., opposite of arrow A1 or arrow A2) causing the top end 30 of the shaft 28 to move through the outer housing 12 away from the distal end of the connector 110, 112. Once the shaft 28 is spaced apart from the connector 110, 112, the practitioner can remove the connector 110, 112 from the outer housing 12 by pulling the male connector 110 axially away from the outer housing 12. By contrast, to release the female connector 112 from the outer housing 12, the practitioner rotates the female connector 112 causing the corresponding threads 128 of the female connector 112 to move away from the threads 44 on the inner surface of the outer housing 12. In some examples, the universal cap 10 of the present disclosure can be a single-use product. As such, the cap 10 can be disposed of after it is removed from the male connector 110 or the female connector 112.

Universal Cap with Inner Shaft

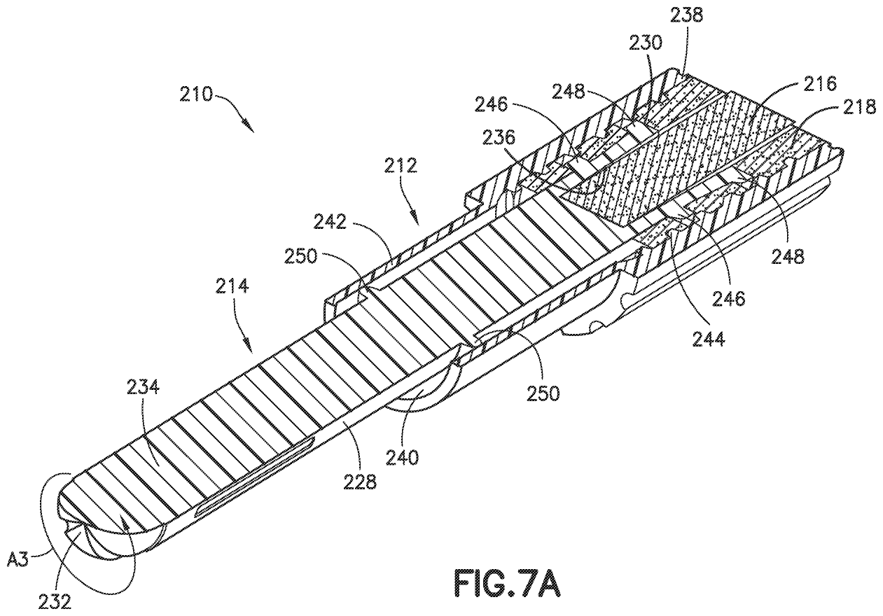

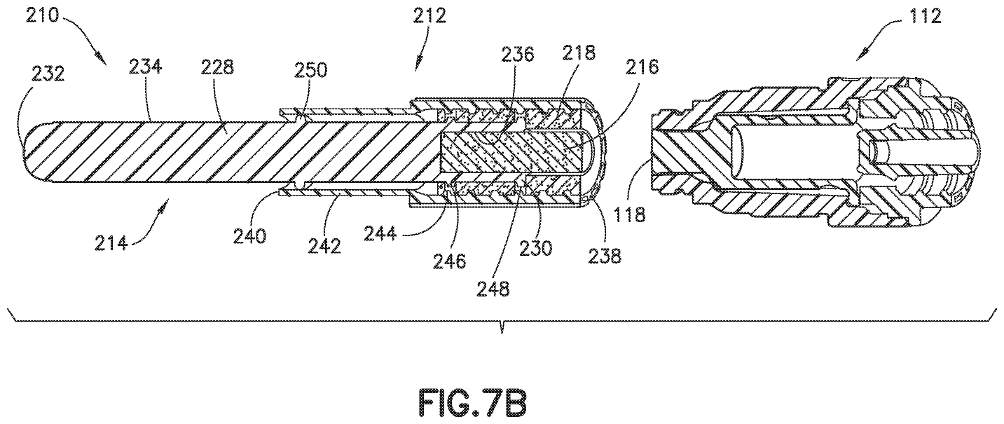

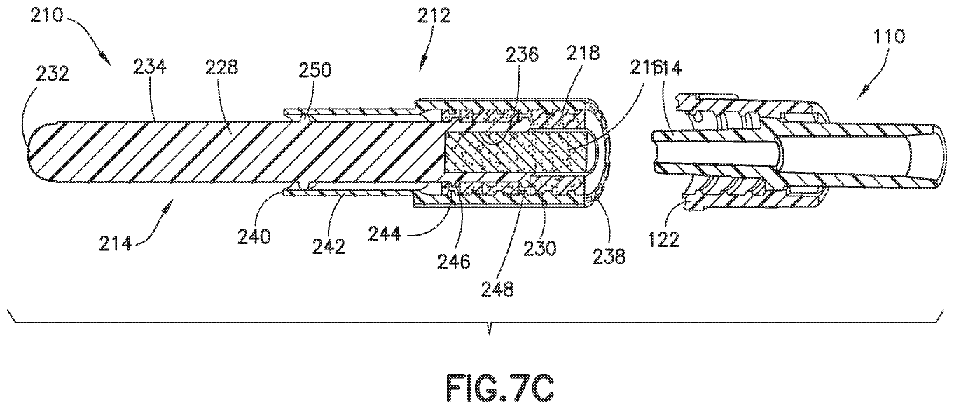

FIGS. 7A-8 show another example of a universal cap 210 configured to be connected to either a male connector 110 (shown in FIG. 7C) or a female connector 112 (shown in FIG. 7B). As in previous examples, the universal cap 210 includes the inner housing 214 and the outer housing 212 positioned over and/or around the inner housing 214. The inner housing 214 comprises the shaft 228 configured to be rotatably received within the outer housing 212. The outer housing 212 comprises an open top end 238, an open bottom end 240, and a sidewall 242 extending between the top end 238 and the bottom end 240. The outer housing 212 also includes the threads 244 configured to engage corresponding threads 128 of the female connector 112. As in previous examples, the inner housing 214 moves through the outer housing 212 as the connectors 110, 112 is being inserted into or removed from the universal cap 210.

The universal cap 210 also includes the cylindrical absorbent member 216 and the tubular absorbent member 218 connected to the inner housing 214. As in previous examples, the absorbent members 216, 218 are configured to contact portions of the male connector 110 and/or the female connector 112, when the male connector 110 or the female connector 112 is inserted into the outer housing 212 for mechanically removing particles, such as microbes, dirt, dust, and other debris, from surfaces of the male connector 110 and/or the female connector 112. The absorbent members 216, 218 include the cleaning or disinfecting solution. As previously described, when the absorbent members 216, 218 are compressed, the cleaning or disinfecting solution is released from the absorbent members 216, 218 and contacts surfaces of the male connector 110 or the female connector 112. As in previous examples, the inner housing 214 also includes a seal 250, such as an O-ring, for sealing against the inner surface of the outer housing 212 for sealing the cleaning or disinfecting solution in the outer housing 212. Specifically, the cleaning or disinfecting solution is retained within a space between an outer surface of the inner housing 214 and the inner surface of the outer housing 212 for cleaning and disinfecting surfaces of the connectors 110, 112 enclosed within the outer housing 212.

The universal cap 210 differs from previous examples in the shape of the inner housing 214. Specifically, unlike in previous examples, the inner housing 214 of FIGS. 7A-8 does not include a cup, base, enlarged grip, or similar structure at the bottom end of the post or shaft. Instead, the inner housing 214 comprises an elongated rod, post, or shaft 228 comprising a first or top end 230, a second or bottom end 232 opposite the top end 230, and an elongated body 234 extending between the top end 230 and the bottom end 232 of the shaft 228. As in previous examples, the shaft 228 is configured to move axially through the outer housing 212 in order to secure the male connector 110 or the female connector 112 to the cap 210.

The shaft 228 also includes structures for engagement with the connectors 110, 112 and with the outer housing 212. For example, the shaft 228 can include the cavity 236, which can be a tapered cavity, extending inwardly from the top end 230 of the shaft 228 sized to receive the stem 114 of the male connector 110. The cavity 236 forms a friction engagement with the tapered outer surface of the stem 114 for connecting the cap 210 to the male connector 110. The shaft 228 also includes the protrusions, such as the upper and lower lugs 246, 248 of previous examples, configured to rotatably engage the threads 244 of the outer housing 212, such that rotation of the shaft 228 (shown by arrow A3 in FIG. 7A) relative to the outer housing 212 draws the shaft 228 through the outer housing 212.

The cap 210 is configured to be connected to both the female connector 112 (shown in FIG. 7B) and the male connector 110 (shown in FIG. 7C). In order to connect the universal cap 210 to the female connector 112, the practitioner inserts a distal end of the female connector 112 through the open top 238 of the outer housing 212. The practitioner may rotate the female connector 112 by one or several rotations causing the threads 128 of the female connector 112 to engage corresponding threads 244 of the outer housing 212. The practitioner then rotates the shaft 228 (shown by arrow A3 in FIG. 7A) causing the top end 230 of the shaft 228 to move towards the distal end and septum 118 of the female connector 112. Moving the shaft 228 towards the female connector 112 causes the absorbent members 216, 218 to compress, which releases cleaning and disinfecting solution from the absorbent members 216, 218. The released cleaning or disinfecting solution contacts surfaces of the female connector 112 for cleaning and disinfecting the surfaces.

In a similar manner, in order to connect the universal cap 210 to the male connector 110, the practitioner first inserts the stem 114 of the male connector 110 through the open top 238 of the outer housing 212, such that the annular shield 122 of the male connector 110 contacts and rests against the open top 238 of the outer housing 212. The practitioner then rotates the shaft 228 (shown by arrow A3 in FIG. 7A) causing the shaft 228 to move through the outer housing 212 bringing the top end 230 of the shaft 228 into contact with the stem 114 of the male connector 110 and, such that, in a fully engaged position, the stem 114 is received within the cavity 236 of the shaft 228. Moving the stem 114 through the outer housing 212 compresses the cylindrical absorbent member 216, releasing the cleaning or disinfecting solution within the outer housing 212. Also, the inner surface of the tubular absorbent member 218 can contact outer surfaces of the stem 114 for cleaning and disinfecting outer surfaces of the stem 114. As in previous examples, the cap 210 can also include the protective cover 254, which is initially provided over the open top end 238 of the outer housing 212, as shown in FIG. 8.

While examples of the universal cap 10, 210 and methods of use of the present disclosure are shown in the accompanying figures and described hereinabove in detail, other examples will be apparent to, and readily made by, those skilled in the art without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are to be embraced within their scope.

The invention claimed is:

1. A universal cap configured to engage at least a first connector and a second connector of different types, comprising:
an inner housing comprising a base comprising an open first end, a closed second end, a sidewall extending therebetween, and a shaft comprising a first end and a second end opposite the first end, which is connected to the closed second end of the base;
an outer housing positioned around the shaft of the inner housing comprising an open first end, an open second end, and a sidewall extending therebetween, wherein an inner surface of the sidewall of the outer housing comprises threads configured to engage corresponding threads of the first connector or the second connector, and wherein the shaft of the inner housing is configured to move through the outer housing toward the first connector or the second connector; and
at least one absorbent member connected to the first end of the shaft configured to contact portions of the first connector or the second connector, when the first connector or the second connector is inserted through the open first end of the outer housing.

2. The cap of claim 1, wherein the first connector is a male needleless connector, and
wherein the shaft of the inner housing is configured to be secured to the male needleless connector by an interference or friction engagement between the shaft and an outer surface of the male needleless connector.

3. The cap of claim 2, wherein the male needleless connector is a male luer connector comprising a stem having a tapered outer surface, and
wherein the shaft of the inner housing comprises a tapered cavity sized to receive the stem of the male luer connector for securing the male luer connector to the cap.

4. The cap of claim 3, wherein the male luer connector further comprises an annular shield extending about the stem, and wherein an open end of the annular shield contacts the open first end of the outer housing as the stem of the male luer connector is inserted through the open first end of the outer housing.

5. The cap of claim 1, wherein the second connector is a female needleless connector, and wherein the threads on the inner surface of the outer housing are configured to engage corresponding threads of the female needleless connector for securing the female needleless connector to the cap.

6. The cap of claim 1, wherein the inner housing and/or the outer housing comprise a rigid thermoplastic polymer, such as polyester, polycarbonate, polypropylene, polyethylene, polyethylene terephthalate, or acrylonitrile butadiene styrene.

7. The cap of claim 1, wherein the shaft comprises a tapered cavity extending inwardly from the first end of the shaft sized to receive a stem of a male luer connector.

8. The cap of claim 1, wherein the inner housing comprises a substantially opaque polymer material.

9. The cap of claim 1, wherein the shaft comprises at least one protrusion configured to rotatably engage the threads of the outer housing, such that rotation of the inner housing relative to the outer housing draws the shaft of the inner housing through the outer housing.

10. The cap of claim 9, wherein the at least one protrusion comprises a plurality of lugs configured to rotatably engage the threads on the inner housing of the sidewall of the outer housing.

11. The cap of claim 1, further comprising an annular seal about the shaft of the inner housing configured to seal against the inner surface of the sidewall of the outer housing preventing cleaning solution from passing through the open second end of the outer housing.

12. The cap of claim 11, wherein the seal is integral with an outer surface of the shaft, or wherein the seal comprises an O-ring positioned about the shaft.

13. The cap of claim 1, wherein the outer housing is configured to move from an initial position, in which the open second end of the outer housing is closer to the open first end of the base than to the closed second end of the base, and a final position, in which the open second end of the outer housing is closer to the closed second end of the base than to the open first end of the base.

14. The cap of claim 1, wherein the outer housing comprises a transparent, substantially transparent, or translucent material, such that the inner housing can be seen through the transparent, substantially transparent, or translucent material of the outer housing.

15. The cap of claim 1, wherein the at least one absorbent member is positioned to cover an opening of the first connector or the second connector when the first connector or the second connector is inserted through the open first end of the outer housing.

16. The cap of claim 1, wherein the at least one absorbent member comprises a cylindrical absorbent member and a tubular absorbent member positioned around the cylindrical absorbent member.

17. The cap of claim 16, wherein the shaft comprises a cavity extending inward from the first end of the shaft, wherein the cylindrical absorbent member is at least partially received within the cavity, and wherein the tubular absorbent member encloses the first end of the cavity and the cylindrical absorbent member, such that a portion of an inner surface of the tubular absorbent member contacts an outer surface of the shaft.

18. The cap of claim 16, wherein the tubular absorbent member comprises at least one opening sized to receive a lug extending outward from the sidewall of the shaft.

19. The cap of claim 16, wherein the cylindrical absorbent member and/or the tubular absorbent member comprise a porous foam comprising a thermoplastic elastomeric material.

20. The cap of claim 16, wherein insertion of the first connector through the open first end of the outer housing causes the first connector to contact the cylindrical absorbent member, and wherein contact between the first connector and the cylindrical absorbent member mechanically removes objects from outer surface(s) of the first connector, or
wherein insertion of the second connector through the open first end of the outer housing causes the second connector to contact an outer surface of the tubular absorbent member, and wherein contact between the second connector and the tubular absorbent member mechanically removes objects from outer surface(s) of the second connector.
